(19) United States
(12) Patent Application Publication
(10) Pub. No.: US 2005/0031618 A1
(43) Pub. Date: Feb. 10, 2005

(54) Induction of tolerance by apoptotic and/or necrotic cells

(76) Inventor: Dror Mevorach, Jerusalem (IL)

Correspondence Address: Martin D Moynihan;Anthony Castorina, 2001 Jefferson Davis Highway, Suite 207, Arlington, VA, 22202, US

(21) Appl. No.: 10/470,536

(22) PCT Filed: Jan. 31, 2002
(86) PCT No.: PCT/IL02/00089

Related U.S. Application Data
(60) Provisional application No. 60/265,127, filed on Jan. 31, 2001.

(51) Int. Cl. A61K 39/395

(52) U.S. Cl. 424/155.1

(57) ABSTRACT

The present invention is directed to a method of inducing tolerance to self-antigens in a subject having an autoimmune diseases. In particular, the invention provides a pharmaceutical composition and method of use thereof for the modulation of immunogical activity in an animal subject. Said modulation may be an increased tolerance to self apoptotic cells, a reduction in the tissue levels of autoantibodies associated with apoptotic cells, a reduction in the tissue levels of autoantibodies associated with an autoimmune disease, a reduction in the level of inflammation and inflammatory mediators associated with an autoimmune disease, a reduction in the level of tissue damage associated with an autoimmune disease, or a combination thereof.

### FIELD OF THE INVENTION

This invention relates to the field of medicine, in particular to the treatment of diseases which arise due to malfunctioning of the immune system, such as autoimmune diseases. The invention relates as well to pharmaceutical compositions comprising apoptotic and/or necrotic cells which are useful for treating such diseases.

### BACKGROUND OF THE INVENTION

The immune system of animals is a complex and multivarious network comprising cells, antibodies, solid and non-solid organs, and chemical messenger molecules which allow for communication between these structures. A hallmark of a healthy immune system is the ability to recognize bacteria, viruses, and other foreign bodies and to effectively attack such pathogens while continuing to distinguish between the foreign bodies and the molecules, cells, tissues and organs comprising the individual organism. When this aspect of an animal's immune system is deficient the result is a state of disease, often one in which the immune system attacks one or more specific molecules or cells leading to tissue and organ damage. Since the immune system destroys bodies recognized as foreign, often known as antigens, through a complex process known as inflammation, of which many different types exist, the immediate and chronic types of tissue damage in autoimmune and inflammatory diseases are frequently the result of one or more types of inflammation.

In autoimmune disease an immune response directed against one or more components of the animal's own tissues or cells results in damage to one or more organs or tissues. In mammals, particularly in humans, many clinically different types of autoimmune disease occur, including subtypes of particular autoimmune disease. Although each type of autoimmune disease is associated with a spectrum of clinical symptoms and aberrant laboratory parameteres, signs and symptoms of autoimmune diseases frequently overlap so that one or more are diagnosed in the same patient. The vast majority cases in which one or more autoimmune disease has been diagnosed are characterized by the presence in the affected subject of autoantibodies. The autoantibodies are directed to one or more molecular or cellular targets, known as antigens, within the animal. Such autoantibodies are present at tissue levels, which are often ten to one hundred times the normal level in healthy individuals and give rise to a significant proportion of the organ and tissue damage associated with the particular autoimmune disease. For example, in the autoimmune disease myasthenia gravis, autoantibodies against a receptor in meuromuscular junction are associated with muscle weakness, while in systemic lupus erythematosus, anti-dsDNA antibodies are associated with nephritis in human patients and can cause nephritis upon injection to normal mice. The tissue and organ damage is attributed to the presence of autoantibodies and to the inflammation, which arises to due inflammatory immune responses, set off by autoantibodies. Thus the signs and symptoms of disease are due to autoantibodies, the autoimmune inflammatory response, or a combination thereof.

Autoimmune diseases include rheumatoid arthritis, graft versus host disease, systemic lupus erythromatosus (SLE), scleroderma, multiple sclerosis, diabetes, organ rejection, inflammatory bowel disease, psoriasis, and other afflictions. It is becoming increasingly apparent that many vascular disorders, including atherosclerotic forms of such disorders, have an autoimmune component, and a number of patients with vascular disease have circulating autoantibodies. Autoimmune diseases may be divided into two general types, namely systemic autoimmune diseases (exemplified by lupus and scleroderma), and organ specific (exemplified by multiple sclerosis, diabetes and atherosclerosis, in which latter case the vasculature is regarded as a specific organ).

One important autoimmune disease is lupus and this disease is a model for enraveling the physiology and developing inventive treatments for autoimmune disease in general. It has long been appreciated that DNA and histones are major autoantigens in systemic lupus erythematosis (SLE). However, only recently has evidence been provided that the DNA-histone complex, i.e., nucleosomes, are the preferred targets of autoantibodies in SLE. The question then arises as to how nucleosomes and several other intracellular antigen targets can be immunogenic in SLE. During apoptosis, the membrane of cells undergoing apoptosis form cytoplasmic blebs, some of which are shed as apoptotic bodies. It was recently demonstrated that exposure of kertinocytes to high frequency light induces apoptosis, and that the cell surface expression of Ro and La, but also of nucleosomes and ribosomes, can be explained by translocation of certain intracellular particles to the apoptotic surface blebs. Significantly, another translocation which occurs during apoptosis is that of phosphatidyl serine (PS), an acidic phospholipid that normally resides on the inside of the cell, but flips to the outside of the cell membrane when the cell undergoes apoptosis. PS like, cardiolipin, is a major autoantigen for anti-phospholipid (aPL) antibodes in SLE. Taken together, these findings provide a unifying hypothesis to explain antigen selection in SLE, e.g., that SLE patients are responding to the exposure of intracellular proteins translocated to the cell surface during apoptosis.

Thus, SLE patients form an immune response to apoptotic material. Although there may be many possible explanations to explain this observation, any explanation must take into account that in SLE patients the uptake of apoptotic cells by macrophages in vitro is reduced. Furthermore, brief, limited administration of syngeneic apoptotic cells to normal strains of mice leads to induction of autoantibodies and glomerular depositions. In addition, it has been shown that the complement system is important in clearance of uptake of apoptotic cells, suggesting the novel hypothesis disclosed herein for the reason why greater than 90% of patients homozygous for C1q and greater than 70% of C4 deficiency patients develop SLE.

This novel understanding of the pathogenesis of SLE may suggest a different approach to the treatment of SLE. Manipulation of the immune system to prevent a deleterious response has been the goal of immunologists for many years in transplantation biology and autoimmune diseases. Traditionally, the main effort was to induce immunosuppression and the current therapy for a classical systemic autoimmune disease such as SLE is drug treatment with corticosteroids, azathyoprine, cyclopohosphamide, and cyclosporine, all of which are administered with the aim suppressing the immune system. Immunosuppression was an important step in ameliorating the 5-years survival rate of SLE patients in the last three decades but it is far from the ideal treatment since no cure is achieved and patients suffer from very serious side effects leading to high rates of morbidity and being the main cause of premature mortality. In that regard, even the newly developed biologics currently under toxicity and efficacy evaluation, such as anti-CD40 ligand, and CTLA4 lg, are non-specific for the autoimmune B and T cell clones and, if successful for autoimmunity, will suppress probably the entire immune system.

In addition to fighting infections, the immune system has other roles in maintaining the normal state of health and function of the animal. Throughout the life span of an animal, tissues become reshaped with areas of cells being removed. This is accomplished by the cells' undergoing a process called programmed cell death or apoptosis, the apoptotic cells disintegrating and being phagocytosed while not becoming disrupted. In many organs, for example, a certain percentage of the cells die off every day while different branches of the immune system are typically called in to remove the dead cells and parts thereof to make room for the new cells which are born to replace them. Were it not for the cellular debri removing cells of the immune system, often known as macrophages, tissue and organ growth would be impossible due to a lack for space.

In fact, the process of apoptosis is considered to be particularly important in the development and maintenance of the immune system itself, where the immune cells which recognize or attack other normal cells of the animal are destroyed and removed by this process. Thus, while apoptosis is a process used by the immune system in protecting the organism, it is also used to maintain tolerance to self antigens and therefore allowing the immune system to fulfill its role in distinguishing the animal's own cells from those of non-self invaders.

Immature dendritic cells (IDC) engulf apoptotic cells and are able to acquire antigens found in the dying cells. IDC that capture apoptotic macrophages infected by killed influenza-virus, mature and activate lymphocytes to mount virus-specific CTL responses in the presence of conditioned media. However, in the absence of infection and conditioned media, IDC do not mature following uptake of apoptotic cells and as a consequence are less able to efficiently present acquired antigens. Furthermore, it has been suggested that following interaction with apoptotic material, IDC may have a role in maintaining peripheral tolerance to self-antigens that are permanently created at different sites. In support of this, autoimmunity or lupus like disease has been observed in mice and human deficient in receptors important for uptake of apoptotic cells such as ABC1 cassette transporter, Mer, and complement deficiencies. Clearance via specific receptors may dictate specific immune response or tolerance as demonstrated by TGF-β and IL-10 secretion by macrophages following uptake by macrophages. So, cytokines, chemokines, eicosanoids, and additional materials found in the milieu of the interaction, may polarize the immune response.

Thus, the aim of the present Invention is to induce tolerance to self antigens in a subject having an autoimmune disease, mainly antigens related to apoptotic and/or necrotic cells.

### BRIEF DESCRIPTION OF FIGURES

FIG. 1 demonstrates the modulation of Immune Response in MRL/MpJ-Fas^{Ipr} Mice Following Injection With Apoptotic Cells vs Placebo—Decrease in anti-single stranded DNA Antibodies

FIG. 2 demonstrates the modulation of Immune Response in MRL/MpJ-Fas^{Ipr} Mice Following Injection With Apoptotic Cells vs Placebo—Decrease in anti-double stranded DNA Antibodies

### SUMMARY OF THE INVENTION

The present invention is directed to a method of inducing tolerance to self-antigens in a subject having an autoimmune disease. According to the present invention, a lack of tolerance to apoptotic and/or necrotic cells is an important aspect in the development of autoimmune disease and an important target for therapy.

In particular, the invention provides a pharmaceutical composition and method of use thereof for the modulation of immunogical activity in an animal subject wherein said modulation is an increased tolerance to apoptotic and/or necrotic cells, a reduction in the tissue levels of autoantibodies associated with apoptotic and/or necrotic cells, a reduction in the tissue levels of autoantibodies associated with an autoimmune disease, a reduction in the level of inflammation and inflammatory mediators associated with an autoimmune disease, a reduction in the level of tissue damage associated with an autoimmune disease, or a combination thereof.

A composition for treating autoimmune diseases according to the present invention should contain antigens, i.e. apoptotic and/or necrotic cells, or fragments thereof, i.e. blebs of apoptotc cells, membrane fragments, and peptides, that upon administration, interact with the immune system of the animal to produce enhanced tolerance to self antigens. In addition, the antigens or fragment thereof should be present in a form which can be recognized by the subject's immune system when the composition is administered to the subject. The desirable antigens may be present on intact apoptotic cells or necrotic cells on fragments thereof.

### DETAILED DESCRIPTION OF THE INVENTION

The present invention provides a pharmaceutical composition and method of use thereof for the modulation of immunogical activity in an animal subject wherein said modulation is an increased tolerance to apoptotic and/or necrotic cells. A composition for treating autoimmune diseases according to the present invention should contain antigens, i.e. apoptotic and/or necrotic cells, or fragments thereof, i.e. blebs of apoptotic cells, membrane fragments, and peptides, that upon administration, interact with the immune system of the animal to produce enhanced tolerance to self antigens.

Antibody isotype switching is a process whereby an immune cell producing one type of antibody isotype directed against a particular antigen begins to produce a second antibody isotype directed against the same antigen. One example of antibody switching occurs in the transition from acute to chronic infection, when a lymphocyte producing an IgG isotype directed an antigen of an infectious invading agent begins to produce an IgM isotype directed against the same antigen, which often signifies the acquisition of an immune state toward the infectious agent. Antibody isotype switching is known to occur in other situations including, but not only, autoimmune states wherein isotype switching occurs for the production of antibodies, i.e. autoantibodies, directed against self-antigens.

According to the present invention, an autoimmune disease is, but not only, one of the following: systemic lupus erythematosis, discoid lupus erythematosis, rheumatoid arthritis, diabetes mellitus, graft versus host disease, scleroderma, multiple sclerosis, diabetes, organ rejection, inflammatory bowel disease, psoriasis, miscarriage, infertility, atherosclerosis, and other inflammatory disorders.

Examples of systemic autoimmune disease are rheumatoid arthritis, lupus and scleroderma, and examples of organ specific autoimmune disease are multiple sclerosis, diabetes and atherosclerosis.

According to the present invention, tolerance is the ability of the immune system to properly recognize self-antigens in a way which does not produce signs or symptoms of an autoimmune disease. The enhanced tolerance to apoptotic and/or necrotic antigens may be obtained by a reduction in the presentation of apoptotic and/or necrotic antigens on dendritic cells.

According to the present invention, administering to a subject a composition comprising apoptotic and/or necrotic cells as antigen is a mean of inducing tolerance and thereby reducing the signs or symptoms of autoimmune disease, in particular a disease associated with a disturbance in the process of apoptosis or clearance of apoptotic and/or necrotic cells.

According to the present invention, a composition to treat autoimmune diseases contains antigens or fragments thereof (peptides) that will enhance the immune mechanism of tolerance to the self-antigens which are associated with the autoimmune disease. Such antigens may be present on apoptotic and/or necrotic cells or released by apoptotic and/or necrotic cells, or a combination thereof. Administering a composition comprising apoptotic and/or necrotic cells is a mean of making available to the immune system of a subject an additional amount of said antigens so as to provide for the development of tolerance to said antigens in the subject. An alternative or parallel explanation for the efficacy of the composition of the present invention in treating an autoimmune disease when administered to a subject afflicted thereby, is that administration of apoptotic and/or necrotic cells invokes an beneficial immune response in addition to that of tolerance induction or enhanced apoptotic cell clearance.

According to the present invention, an autoimmune response is an immune response directed against one or more self-antigens of an animal.

According to the present invention, apoptosis is a process of programmed cell death, wherein the cell enters a stage characterized by the breakdown or disappearance of cellular components essential to maintainance of the normal differentiated state of the cell, while maintaining an intact, non-porous membrane. In one aspect of apoptosis, the cell undergoes a process of de-differentiation whereby the ability to remain in a functional, viable, differentiated state is lost. The pathway of apoptosis may include, but not only, loss of membrane potential of the mitochondria, activation of serine proteases, activation of caspases, activation of other proenzymes, cleavage of proteins, cleavage of DNA, cleavage of DNA to form nucleosomes, phosphorylation of proteins, exposure of phosphatidylserine residues on the outer membrane surface, the appearance of blebs, condensed chromatin, contracted cytoplasm, or a combination thereof. Of the foregoing, early signs of apoptosis include, but not only, loss of membrane potential of the mitochondria, cleavage of proteins, cleavage of DNA, and protein phosphorylation and exposure of phosphatidylserine. Nectosis is the feature of cells undergoing death that leads among other things to disruption of the membrane and swelling of the cell.

In one embodiment, the present invention provides a method of treating a subject having an autoimmune disease, comprising the steps of: obtaining cells from the subject; inducing cell death in said cells resulting in apoptotic and/or necrotic cells; administering to the subject an amount of said apoptotic and/or necrotic cells effective to produce a modified immune response in said subject, thereby treating the subject with the autoimmune disease.

In another embodiment, the present invention provides a method of treating a subject having an autoimmune disease, comprising the step of administering to the subject an amount of apoptotic and/or necrotic cells effective to produce a modified immune response in said subject, thereby treating the subject with the autoimmune disease.

According to the present invention, an apoptosis-inducing agent is a natural or synthetic molecule, a natural or synthetic antibody, an immunological cell, gamma- or UV-irradiation, in the presence of which, or upon contact by which, a cell becomes an apoptotic cell. Examples of apoptosis-inducing agents are immunosuppressive drugs including, but not only, corticosteroids, cyclophosphamide, methotrexate, azothioprine, cyclosporine, staurosporine, or a combination thereof. Necrosis can be induced by H₂O₂ or heating or using other methods known in the art.

According to the present invention, an apoptosis and/or necrosis inducing treatment is a set of one or more environmental conditions in which a cell becomes an apoptotic and/or necrotic cell. Examples of apoptosis-inducing treatments are U.V.- or gamma-irradiation, heating, cooling, serum deprivation, growth factor deprivation, acidifying, diluting, alkalizing, ionic strength change, serum deprivation, irradiating, or a combination thereof.

According to the present invention, induction of apoptosis occurs when a cell becomes an apoptotic cell. For induction of apoptosis in any particular cell or cells, the choice of apoptosis-inducing agent and apoptosis-inducing treatment to yield an apoptotic cell or cells is a function of the type of cell, tissue of origin, and means of obtaining the cell sample. Apoptosis may be induced in vivo, in situ, in vitro, or ex vivo. Some of the commonly known apoptosis-inducing methods are contacting a cell with a steroid, such as dexamethasone, exposing a cell to radiation, in particular gamma-radiation, exposing a cell to conditions of serum deprivation, in particular 0-5% serum, contacting a cell with perforin, or a combination thereof. In addition to inducing apoptosis, many of these agents and conditions produce as well, in varying proportions, induction of necrosis, otherwise known as accidental cell death, necrotic cells (primary or secondary), and otherwise damaged, non-apoptotic cells. Thus, for purposes of the present invention, apoptotic cells may comprise at any one time both apoptotic cells and fragments thereof, and as well a certain percentage of necrotic or lysed cells and fragments thereof.

The presence of an apoptotic cell may be confirmed by DNA electrophoresis, TUNEL (DNA labeling), annexin-FITC plus propidium iodide, propidium iodide staining of fragmented DNA (hypodipoid region), caspase activation, cleavage of target proteins, morphologically using light microscopy with appropriate staining, electron microscopy, or a combination thereof.

According to the present invention, an apoptotic cell is a cell in which apoptosis has been induced either through the course of a disease process or induced through contact with an apoptosis-inducing agent, exposure to an apoptosis-inducing treatment, or a combination thereof.

According to the present invention, apoptotic and/or necrotic cells are preferably obtained through a method comprising the steps of: obtaining cells concentrated from blood; contacting the cells with an apoptosis-inducing agent or with a necrosis-inducing agent and incubating at 37° C. in a physiologically suitable medium.

According to the present invention, an apoptosis-inducing agent is a natural or synthetic molecule, a natural or synthetic antibody, an immunological cell, gamma- or UV-irradiation, in the presence of which, or upon contact by which, a cell becomes an apoptotic cell. Examples of apoptosis-inducing agents are immunosuppressive drugs including, but not only, corticosteroids, cyclophosphamide, methotrexate, azothioprine, cyclosporine, staurosporine, or other compounds or a combination thereof. Necrosis can be induced by H₂O₂ or heating or with other methods known in the art.

According to the present invention, an apoptosis- and/or necrosis-inducing treatment is a set of one or more environmental conditions in which a cell becomes an apoptotic and/or necrotic cell. Examples of apoptosis-inducing treatments are U.V.- or gamma-irradiation, heating, cooling, serum deprivation, growth factor deprivation, acidifying, diluting, alkalizing, ionic strength change, serum deprivation, irradiating, or a combination thereof.

According to the present invention, induction of apoptosis occurs when a cell becomes an apoptotic cell. For induction of apoptosis in any particular cell or cells, the choice of apoptosis-inducing agent and apoptosis-inducing treatment to yield an apoptotic cell or cells is a function of the type of cell, tissue of origin, and means of obtaining the cell sample. Apoptosis may be induced in vivo, in situ, in vitro, or ex vivo. Some of the commonly known apoptosis-inducing methods are contacting a cell with a steroid, such as dexamethasone, exposing a cell to radiation, in particular gamma-radiation, exposing a cell to conditions of serum deprivation, in particular 0-5% serum, contacting a cell with perforin, or a combination thereof. In addition to inducing apoptosis, many of these agents and conditions produce as well, in varying proportions, induction of necrosis, otherwise known as accidental cell death, necrotic cells (primary or secondary), and otherwise damaged, non-apoptotic cells. Thus, for purposes of the present invention, apoptotic cells may comprise at any one time both apoptotic cells and fragments thereof, and as well a certain percentage of necrotic or lysed cells and fragments thereof.

The presence of an apoptotic cell may be confirmed by DNA electrophoresis, TUNEL (DNA labeling), annexin-FITC plus propidium iodide, propidium iodide staining of fragmented DNA (hypodipoid region), caspase activation, cleavage of target proteins, morphologically using light microscopy with appropriate staining, electron microscopy, or a combination thereof.

According to the present invention, an apoptotic cell is a cell in which apoptosis has been induced either through the course of a disease process or induced through contact with an apoptosis-inducing agent, exposure to an apoptosis-inducing treatment, or a combination thereof.

In one aspect of the composition and methods of the invention, the cells are derived from the subject to be treated (autologous source), so as to avoid the possibility of contamination with undesirable infectious agents which may be present in donors. Thus, in one embodiment, the composition comprises apoptotic cells which are derived from cells obtained from the subject to be treated. For obtaining cells, virtually any technique for obtaining cells may be used, with the exception of procedures which markedly interfere with the potential for a cell to become an apoptotic cell or which interfere with the potential of an apoptotic cell to induce tolerance. Furthermore, the use of an autologous source of cells is preferable since apoptotic cells obtained therefrom are most likely to invoke the desired response of tolerance and are most likely to contain the desired antigen configuration.

Thus, a therapeutic composition comprising apoptotic and/or necrotic cells which are able to exert a strong influence on the immune system and encourage the development of tolerance. For purposes of the present invention, the apoptotic cells may be rendered more highly active through modifications which enhance immunogenecity. Thus antigenic apoptotic cells may have a more avid interaction with the immune system when administered with an immunosuppressing molecules such as IL-10 or other immunosuppressing cytokines, chemokines or other peptides or molecules. Other procedures which are known to enhance immunogenecity are linking together, i.e. cross-linking, or linking with other ligands directly or through spacers.

According to the present invention, an apoptotic and/or necrotic cell may derive from any body tissue, soft tissue, solid tissue, lymphatic tissue or hematogenous tissue. The cell may be of syngeneic origin or pedigree, autologous origin or pedigree, allogenic origin or pedigree, xenogenic origin or pedigree or a combination thereof. An apoptotic and/or necrotic cell may be derived from a cell obtained from a body tissue, including but not only, the following: blood, sputum, lymph, lymph node, thymus, bone marrow, saliva, dermis, epidermis, hypodermis, mucosa, submucosa, an internal organ, connective tissue, muscle, smooth muscle, synovial fluid, spinal fluid, or a combination thereof.

According to the present invention, for the purpose of obtaining cells, a cell or cells may be obtained from a body tissue by a tissue biopsy; an exfoliative biopsy; a fine needle biopsy; a blood extract; a bone marrow tap; a lymph node biopsy; a lymph node aspirate; a thymus biopsy; a thymus aspirate; a synovial fluid aspiration; a bronchial lavage; a peritoneal lavage; a peritoneal tap; a pleural tap; a spinal fluid tap; a body tissue stored ex vivo; a tissue culture; a skin biopsy; a scraping; an exfoliation; a mucosal biopsy; a mucosal scraping; a cell culture; a cultured cell line; a cultured cell line comprising apoptotic cells; a cultured cell line comprising a gene library; transformed cells; transgenic cells; a cell modified through insertion of a transgene; or a combination thereof. Cells may be of syngeneic origin or pedigree, autologous origin or pedigree, allogenic origin or pedigree, xenogenic origin or pedigree, or a combination thereof.

According to the present invention, a transformed cell is a cell, or an ancestor thereof, into which has been introduced, by means of recombinant DNA techniques, a DNA molecule encoding a desired gene.

According to the present invention, a transgene is any piece of DNA which is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism.

According to the present invention, a transgenic cell is a cell which includes a DNA sequence which is inserted by artifice into the cell and becomes part of the genome of the organism which develops from that cell. As used herein, the transgenic organisms are generally transgenic mammalian (e.g., rodents such as rats or mice) and the DNA (transgene) is inserted by artifice into the nuclear genome.

According to the present invention, elements of the immune system of an animal include, but not only, the following: antibodies, chemokines, eukocytes, lymphocytes, T-cells, B-cells, plasma cells, granulocytes, neutrophils, macrophages, monocytes, eosinophils, platelets, dendritic cells, antigen presenting cells, or a combination thereof.

According to the present invention, a modified immune response is a change in the amount, level, rate of synthesis, rate of degradation, pattern of distribution, systemic concentration, localized concentration of one or more elements of the immune system of the organism in one or more tissues of the organism.

According to the present invention, modulation of an immune response is an action which results in a modified immune response.

According to the present invention, modulation of an autoimmune response is an action which results in a modified autoimmune response.

According to the present invention, treatment of a disease is an action which results in a reduction in the severity of one or more signs or symptoms of said disease including, but not only, a reduction in the levels of autoantibodies, a reduction in the levels of inflammatory mediators, a reduction in inflammation, a reduction in tissue damage, subjective relief from any symptom attributed to the disease including subjective relief from pain or discomfort reported by the subject, or a combination thereof.

Method of Treatment

In one embodiment, the present invention provides a method of treating a subject having an autoimmune disease, comprising the steps of: obtaining cells from the subject; inducing cell death in said cells resulting in apoptotic and/or necrotic cells; administering to the subject an amount of said apoptotic and/or necrotic cells effective to produce a modified immune response in said subject, thereby treating the subject with the autoimmune disease.

In another embodiment, the present invention provides a method of treating a subject having an autoimmune disease, comprising the step of administering to the subject an amount of apoptotic and/or necrotic cells effective to produce a modified immune response in said subject, thereby treating the subject with the autoimmune disease.

In one embodiment, induction of cell death is obtained by exposing said cells to an apoptosis-inducing agent, apoptosis-inducing treatment, a necrosis-inducing agent or a necrosis-inducing treatment

In one embodiment, the modified immune response is an increased tolerance to self-apoptotic cells or a reduction in the tissue level of auto-antibodies associated with self-apoptotic cells. Said auto-antibodies may be antinuclear antibodies, anti-single stranded DNA antibodies, anti-double stranded DNA antibodies, anti-cardiolipin antibodies, anti-phosphatidylserine antibodies, anti-2GPI antibodies, anti-Sm antibodies, anti-RNP antibodies, or anti-Ku antibodies.

In another embodiment, the modified immune response in said subject is a reduction in the level of inflammatory response. Said inflammatory response may be associated with chemokines, cytokines, eicosanoids, complement proteins, C-reactive protein, TNF, or a combination thereof.

In another embodiment, the autoimmune disease is associated with an immune response to self-antigens appearing on apoptotic cells. Said autoimmune disease may be systemic or discoid lupus, erythematosis, rheumatoid arthritis, polymyositis, or vasculitis.

In one embodiment, the cells of the invention are derived from autologous origin. Said cells may be derived from hematopoetic cells, thymocytes, splenocytes, lymphocytes, monocytes, or a combination thereof. In another embodiment, the cells of the invention may be derived from any other sources such as cell lines.

In another embodiment, the amount of said composition comprising apoptotic cells is at least one hundred apoptotic cells of a pharmaceutically acceptable fragment thereof, per kg body weight, in combination with a pharmaceutically acceptable carrier.

In one embodiment, the apoptosis-inducing agent is a steroid, a peptide, a protein, a sugar, a lipid, an antibody, or a combination thereof. Said steroid may be for example dexamethasone. Said protein may be for example perforin.

In another embodiment, the apoptosis-inducing treatment is cooling, heating, acidifying, diluting, alkalizing, ionic strength changing, serum deprivating, irradiating, or a combination thereof.

In one embodiment of the method of the invention, the composition comprising apoptotic cells is administered intravenously, intradermally, subdermally, intramuscularly, orally or a combination thereof. The composition may be administered in combination with an immunosuppressing molecules such as IL-10 or TGF-β.

In a preferred embodiment of the method of the invention, the cells are obtained from the blood of the subject to be treated through separation of said blood into fractions, resuspension of the fraction containing the desired cells in a physiologically acceptable buffered medium, adding an apoptosis-inducing agent under conditions which induce apoptosis, such as an apoptosis-inducing treatment while maintaining the pH, ionic strength, and temperature at physiologically acceptable limits to form a composition comprising apoptotic cells; the composition is injected into the subject via an effective intra- or extravascular route in an amount of between 500,00 to 50×10⁹ cells per 70 kg human subject at a frequency of once or twice per day until the desired modified immune response is obtained, thereby treating the subject.

Pharmaceutical Composition

The invention provides a pharmaceutical composition comprising an effective amount of apoptotic and/or necrotic cells, wherein administration of said composition to a subject produces a modified immune response in said subject.

According to the present invention, a pharmaceutical composition comprising apoptotic cells may be obtained by subjecting said cells to an apoptosis-inducing agent, an apoptosis-inducing treatment, or a combination thereof.

In one embodiment, a composition comprising apoptotic and/or necrotic cells is produced by contacting said cells with an apoptosis-inducing agent or necrosis-inducing agent. In another embodiment, a composition comprising apoptotic and/or necrotic cells is produced by exposing said cells to an apoptosis-inducing treatment or necrosis-inducing treatment. In another embodiment, a composition comprising apoptotic cells is produced by exposing said cells to an apoptosis-inducing agent before, during, or following exposure of said cells to an apoptosis-inducing treatment.

In one embodiment, said composition comprises at least one hundred apoptotic and/or necrotic cells. In one embodiment, said composition comprises from between one hundred to five billion cells.

In one embodiment of the composition of the invention, said modified immune response is increased tolerance to self-apoptotic cells. In another embodiment of the composition of the invention, said modified immune response is a reduction in the tissue levels of autoantibodies in said subject.

In another embodiment of the composition of the invention, said autoantibodies are anti-nuclear antibodies, anti-single stranded DNA antibodies, anti-double stranded DNA antibodies, anti-cardiolipin antibodies, anti-phosphatidylserine antibodies, anti-2GPI antibodies, anti-Sm antibodies, anti-RNP antibodies, anti-Ku antibodies, or a combination thereof.

In another embodiment of the composition of the invention, said modified immune response is a reduction in the level of inflammation or tissue damage, or a combination thereof, in said subject.

In another embodiment of the composition of the invention, said inflammatory response is associated with chemokines, cytokines, eicosanoids, complement proteins, C-reactive protein, TNF, dendritic cells or a combination thereof.

In another embodiment of the composition of the invention, said autoimmune disease is associated with an immune response to self-antigens appearing on apoptotic cells.

In another embodiment of the composition of the invention, said autoimmune disease may be systemic or discoid lupus erythematosis, rheumatoid arthritis, polymyositis, or vasculitis.

In another embodiment of the composition of the invention, the cells are derived from hematopoetic cells, thymocytes, splenocytes, lymphocytes, monocytes, cell lines or a combination thereof.

In another embodiment of the composition of the invention, said apoptosis-inducing agent is an immunosuppressive medication, including, but not only, the following: azathioprine, cyclophosphamide, methotrexate, prednisone, cyclosporine, or a combination thereof.

In another embodiment of the composition of the invention, said apoptosis-inducing treatment is cooling, heating, acidifying, diluting, alkalizing, ionic strength change, serum deprivation, irradiating, or a combination thereof.

In another embodiment of the composition of the invention, said composition is suitable for administration via an intravenous route, an intradermal route, a subdermal route, an intramuscular route, or a combination thereof.

In another embodiment of the composition of the invention, said composition is administered in combination with immunosupressing molecules such as IL-10 or TGF-β.

The composition of the invention may be administered with a pharmaceutically-acceptable diluent, carrier, or excipient, in unit dosage form. Conventional pharmaceutical practice may be employed to provide suitable formulations or compositions to administer the composition to patients having an autoimmune disease. Any appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions, prepared fresh or from lyophilized cells. Methods well known in the art for making formulations are found in, for example, “Remington's Pharmaceutical Sciences.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer lactide/glycolide copolymer, or polyoxyethylene-polyoxypropylene copolymers may be used to control the release of the compounds. Other potentially useful parenteral delivery systems for composition comprising apoptotic cells include ethylene-vinyl acetate copolymer particles, osmotic pumps, implantable infusion systems, and liposomes.

If desired, treatment with the composition of the invention may be combined with more traditional therapies for the disease such as surgery, radiation, or chemotherapy for cancers; surgery, steroid therapy, immunosuppresion therapy and chemotherapy for autoimmune diseases or graft vs host disease; antiviral therapies for AIDS; and for example, tissue plasminogen activator for ischemic injury.

### Experimental Results

In the study presented herein, one of the classical models for SLE-like disease, the MRL/MpJ-Fas^{Ipr}, was used for tolerance induction to self-apoptotic cells. These mice develop SLE-like disease due to mutation in Fas, a receptor that mediates apoptosis and activation of induced cell death of the immune system. Since in SLE patients, as well as in MRL/MpJ-Fas^{Ipr} mice, the development of autoantibodies and kidney disease are the most specific pathophysiological parameters, those parameters were evaluated in MRL/MpJ-Fas^{Ipr} following induction of tolerance to self apoptotic cells.

### Methods and Materials

Immunization protocol. MRL/MpJ-Fas^{Ipr} and C3H-SnJ mice were obtained from Jackson Laboratories, Bar Harbor, Me. Thymocytes and splenoscytes were prepared from 4 to 8 week-old mice as known in the art. A composition comprising sex- and age-matched syngeneic apoptotic cells was injected at 5×10⁶ cells per mouse and compared to syngeneic, sex- and age-matched mice that were injected with the vehicle (saline). The route of administration ws i.v., via the tail vein, without further manipulation. The cells were incubated at 37° C. in 5% CO₂ for 1 to 3 hours to allow apoptotic changes to occur. After incubation, the apoptotic cells were injected into each mouse recipient. The injections were performed every week for a total of four to six injections.

Apoptotis. Apoptotsis of thymocytes or splenocytes was induced by either serum deprivation, 1 micromolar dexamethasone, or gama-irradiation (66 rad). Apoptosis was confirmed by annexin-FITC staining by flow cytometry, DNA fragmentation and propidium iodide staining of fragmented DNA.

Immune response. Serum samples were obtained immediately prior to immunization and at two-weeks intervals following immunization. The immune response was evaluated by quantifying serum anti-ssDNA and anti-dsDNA by ELISA as known in the art. Sera were diluted 1:100 for the autoantibody screens.

Clinical and pathological evaluation. Mice were examined every day for clinical signs of disease and once a month for hematuria or proteinurea. After four months the mice were killed and the kidneys examined histologically and using fluorescent immune staining.

### Results

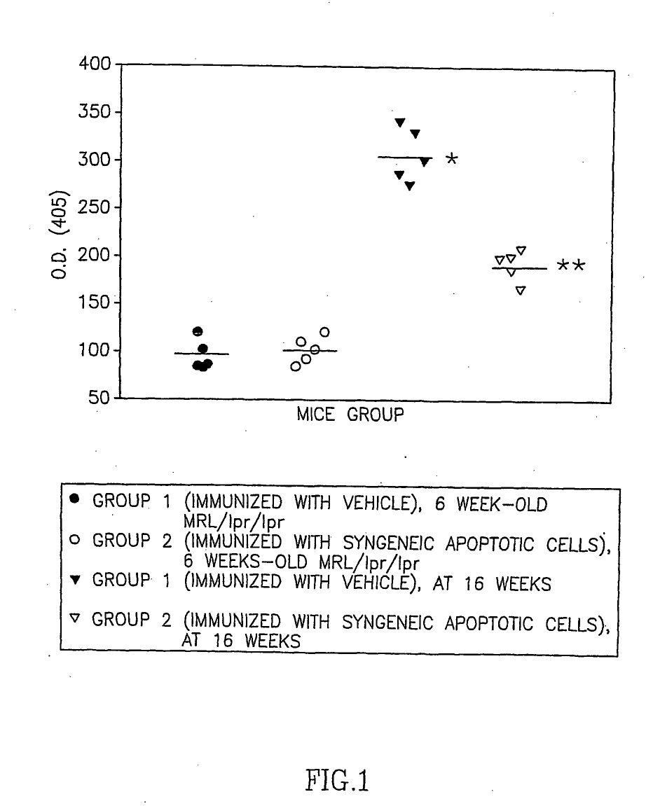

Two groups of age- and sex-matched MRL/MpJ-Fas^{Ipr} mice were compared. In group 1, 200 microliter of saline containing syngeneic apoptotic cells were i.v. injected into each of one of five mice in a weekly interval for five times. In group 2, 200 microliter of saline (the vehicle for the first group) were injcted to the same number of mice at the same time. IgG anti-ssDNA O.D. levels were conseutivly measured in two weeks intervals and were comparable to the level before immunization, mean O.D. of 0.096±0.018 in both groups (FIG. 1). When compared 10 weeks following the beginning of the immunization, mice immunized with vehicle alone had, as expected from mice that developed lupus-like disease, higher levels, 0.308±0.029 (p<0.0000, student t-test). However, mice injected with 1×10⁶ syngeneic apoptotic cells had significantly reduced levels of autoantibodies, 0.193±0.017 (p<0.0000, student t-test). In FIG. 1: ? Group 1 (immunized with vehicle), 6 weeks-old MRL/Ipr/Ipr ; 0 Group 2 (immunized with syngeneic apoptotic cells), 6 weeks-old MRL/Ipr/Ipr; \check{} Group 1 (immunized with vehicle), at 16 weeks; ? Group 2 (immunized with syngeneic apoptotic cells), at 16 weeks.

In order to evaluate the increase in anti-ssDNA, serial bimonthly sera samples were evaluated simultaneously and showed for IgM, 0.198±0.017, 0.205±0.02, and 0.300±0.033 for IgM; 0.378±0.037 for mice immunized with saline; and 0.108(+0.03), 0.170(+0.07), 0.186(+0.04) and 0.203(+0.8) for mice immunized with apoptotic cells. The O.D. statistical evaluation showed that this decrease did not reach significance. In contrast, IgG anti-ssDNA levels became significantly decreased following the immunization with syngeneic apoptotic cells: 0.132±0.09, 0.196±0.019, 0.244±0.022, and 0.308±0.029 for mice immunized with saline, vs. 0.109±0.012 (p=non-significant), 0.129±0.15, p<0.04), 0.166±0.014, (p<0.04), 0.192±0.17) (p<0.01), for mice immunized with syngeneic apoptotic thymocytes. As shown in FIG. 1, at age 16 weeks, a marked decrease in anti-ssDNA was noted in all mice immunized with syngeneic apoptotic cells.

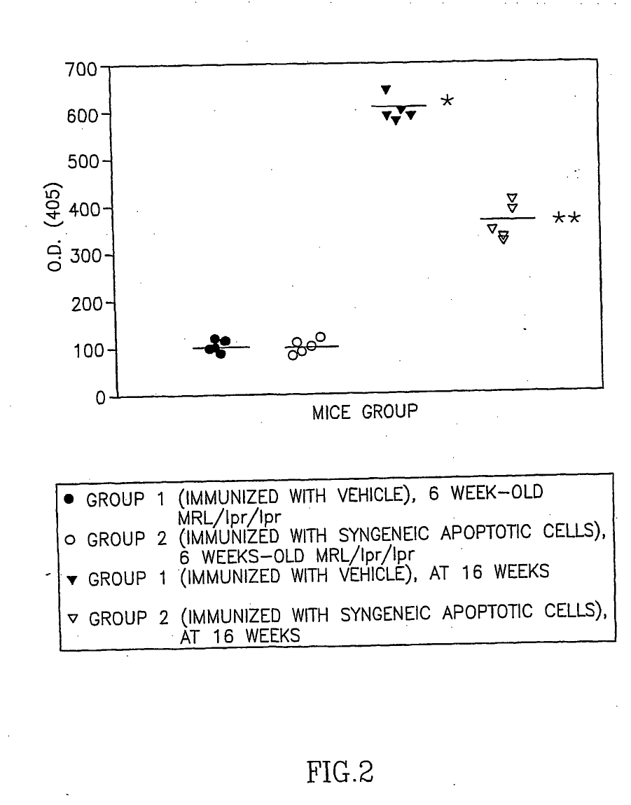

In order to see if autoantibodies even more specific for SLE were decreased, anti-dsDNA was measured in all mice at the age of 6 weeks, before starting to immunize, and at 16-18 weeks of age, upon sacrifice. As shown in FIG. 2, anti-dsDNA was significantly reduced (p<0.00) in mice immunized with syngeneic apoptotic cells. Anti-dsDNA in average of 0.599±0.026 measured in mice injected with saline and 0.358±0.038 in average in mice injected with syngeneic apoptotic cells. In FIG. 2: ? Group 1 (immunized with vehicle), 6 weeks-old MRL/Ipr/Ipr; 0 Group 2 (immunized with syngeneic apoptotic cells), 6 weeks-old MRL/Ipr/Ipr; \check{} Group 1 (immunized with vehicle), at 16 weeks; ? Group 2 (immunized with syngeneic apoptotic cells), at 16 weeks.

To further compare if the clinical response follows the serological one, kidney-disease was compared in the two groups. None of the mice had any evidence for proteinuria or hematuria as measured by urine-stick at 6 weeks of age, before the immunization. At 16 weeks, mice immunized with saline had significant elevations in proteinuria and hematuria as demonstrated in Table 1. At 16 weeks all mice injected with saline alone demonstrated glomerular disease manifested by proteinuria and hematuria. However, mice injected with syngeneic apoptotic cells showed marked improvement (Table 1) consistent with the serological response. In two out of five, no deterioration or very slight deterioration was noticed. In Table 1, Ipr-Apo=MRL/MpJ-Fas^{Ipr} mouse injected with syngeneic apoptotic cells; Ipr-S=MRL/MpJ-Fas^{Ipr} mouse injected with saline; and, C3H/SnJ is a normal mice used for control.

In order to confirm the clinical response, the extent of the disease progression in the kidneys were evaluated by paraffin embedded and immunofluorescent histological studies. Table 2 summarizes the histopathological findings in blindly chosen three kidney sections of each group and demonstrates that mice injected with syngeneic apoptotic cells showed decreased involvement of disease in the glomeruli, vessels and in the tubuli. In Table 2, Ipr-Apo=MRL/MpJ-Fas^{Ipr} mouse injected with syngeneic apoptotic cells; Ipr-S=MRL/MpJ-Fas^{Ipr} mouse injected with saline; and, C3H/SnJ is a normal mice used for control.

TABLE 1
Proteinuria and Hematuria in MRL/MpJ-Fas^{Ipr} Mice Injected with
Syngeneic Apoptotic Cells

| | |
|---|---|
| Proteinuria | Hematuria |

| Mice | 6 weeks | 16 weeks | 6 weeks | 16 weeks |
|---|---|---|---|---|
| C3H/SnJ | +1 | +1 | 0 | 0 |
| Ipr-S | +1 | +2 | 0 | +2 |
| Ipr-S | +1 | +3 | 0 | +1 |
| Ipr-S | +1 | +2 | 0 | +3 |
| Ipr-S | +1 | +2 | 0 | +2 |
| Ipr-S | +1 | +3 | 0 | +1 |
| Ipr-Apo | +1 | +2 | 0 | +1 |
| Ipr-Apo | +1 | +1 | 0 | +1 |
| Ipr-Apo | +1 | +1 | 0 | +1 |
| Ipr-Apo | +1 | +1 | 0 | 0 |
| Ipr-Apo | +1 | +2 | 0 | +1 |

TABLE 2
Histological and Indirect Immunofluoresence Evaluation for IgG
Deposits in MRL/MpJ-Fas^{Ipr}

| | Fluoresence | Histology | | | Indirect |
|---|---|---|---|---|---|
| | Tubuli | GN | Vessels | Tubuli | GN |
| C3H/SnJ | — | — | — | — | — |
| Ipr-S | +2 | +3 | +1 | +3 | +3 |
| Ipr-S | +2 | +2 | +1 − 2 | +4 | +3 |
| Ipr-S | +2 | +2 − 3 | +0 − 1 | +3 | +2 |
| Ipr-S | +1 | +2 | +0 | +1 | +1 |
| Ipr-S | +2 | +1 | +0 | +2 | +1 |
| Ipr-S | +1 | +1 | +1 | +3 | +1 |

1. A method of treating a subject having an autoimmune disease, comprising the steps of:
obtaining cells from the subject;
inducing cell death in said cells resulting in apoptotic and/or necrotic cells;
administering to the subject an amount of said apoptotic and/or necrotic cells effective to produce a modified immune response in said subject, thereby treating the subject with the autoimmune disease.

2. A method of treating a subject having an autoimmune disease, comprising the step of administering to the subject an amount of apoptotic and/or necrotic cells effective to produce a modified immune response in said subject, thereby treating the subject with the autoimmune disease.

3. The method of claim 1 whereby the step of inducing cell death is obtained by exposing said cells to an apoptosis-inducing agent or to a necrosis-inducing agent.

4. The method of claim 1 whereby the step of inducing cell death is obtained by exposing said cells to an apoptosis-inducing treatment or to a necrosis-inducing treatment.

5. The method according to any one of claims 1 to 4, whereby said modified immune response is an increased tolerance to self-apoptotic cells.

6. The method according to claim 5, whereby said modified immune response in said subject is a reduction in the tissue level of auto-antibodies associated with self-apoptotic cells.

7. The method according to claim 6, whereby said auto-antibodies are anti-nuclear antibodies, anti-single stranded DNA antibodies, anti-double stranded DNA antibodies, anti-cardiolipin antibodies, anti-phosphatidylserine antibodies, anti-2GPI antibodies, anti-Sm antibodies, anti-RNP antibodies, or anti-Ku antibodies.

8. The method according to claim 5, whereby said modified immune response in said subject is a reduction in the level of inflammatory response.

9. The method according to claim 8, whereby said inflammatory response is associated with chemokines, cytokines, eicosanoids, complement proteins, C-reactive proteins, TNF, dendritic cells or a combination thereof.

10. The method according to claim 1 or 2, whereby said autoimmune disease is associated with an immune response to self-antigens appearing on apoptotic cells.

11. The method according to claim 10, whereby said autoimmune disease is systemic or discoid lupus, erythematosis, rheumatoid arthritis, polymyositis, or vasculitis.

12. The method according to claim 1 or 2, whereby said cells are hematopoetic cells, thymocytes, splenocytes, lymphocytes, monocytes, a cultured cell line or a combination thereof.

13. The method according to claim 3, whereby said apoptosis-inducing agent is a steroid, a peptide, a protein, a sugar, a lipid, an antibody, or a combination thereof.

14. The method according to claim 13, whereby said steroid is dexamethasone.

15. The method according to claim 13, whereby said protein is perforin.

16. The method according to claim 1 or 2, whereby said composition is administered in combination with an immunosuppressing molecules.

17. A pharmaceutical composition comprising an effective amount of apoptotic and/or necrotic cells, whereby the administration of said composition to a subject suffering from an autoimmine disease produces a modified immune response in said subject.

18. The composition according to claim 17, wherein said modified immune response is a reduction in the tissue level of auto-antibodies associated with apoptotic cells in said subject.

19. The composition according to claim 18, wherein said auto-antibodies are anti-nuclear antibodies, anti-single stranded DNA antibodies, anti-double stranded DNA antibodies, anti-cardiolipin antibodies, anti-phosphatidylserine antibodies, anti-2GPI antibodies, anti-Sm antibodies, anti-RNP antibodies, anti-Ku antibodies, or a combination thereof.

20. The composition according to claim 17, wherein said modified immune response is a reduction in the level of inflammatory response.

21. The composition according to claim 20, wherein said inflammatory response is associated with chemokines, cytokines, eicosanoids, complement proteins, C-reactive proteins, TNF, dendritic cells or a combination thereof.

22. The composition according to claim 17, wherein said autoimmune disease is associated with an immune response to self-antigens appearing on apoptotic cells.

23. The composition according to claim 17, wherein said cells are from autologous origin.

24. The composition according to claim 23, wherein said cells are hematopoetic cells, thymocytes, splenocytes, lymphocytes, monocytes, or a combination thereof.

25. The composition according to claim 17, whereby said apoptotic and/or necrotic cells are obtained by contacting the cells with an apoptosis-inducing agent or a necrotic-inducing agent or by exposing the cells to an apoptosis-inducing treatment or a necrosis-inducing treatment, or a combination thereof.

26. The composition according to claim 25, wherein said apoptosis-inducing agent is a steroid, a peptide, a protein, a sugar, a lipid, an antibody, or a combination thereof.

27. The composition according to claim 26, wherein said steroid is dexamethasone.

28. The composition according to claim 26, wherein said protein is perforin.

29. The composition according to claim 17, wherein said composition is suitable for administration via an intravenous route, an intradermal route, a subdermal route, an intramuscular route, oral administration or a combination thereof.

30. The composition according to claim 17, wherein said composition is administered in combination with an immunosuppressing molecules.
